(19) United States
(12) Patent Application Publication
(10) Pub. No.: US 2026/0207216 A1
(43) Pub. Date: Jul. 23, 2026

(54) DEVICE, ARRANGEMENT AND METHOD FOR REMOVING TISSUE PARTICLES

(71) Applicant: Erbe Elektromedizin GmbH, Tuebingen (DE)

(72) Inventors: Frank STRAUB, Reutlingen (DE); Nermin SALKIC, Moessingen (DE); Andreas FECH, Tuebingen (DE)

(21) Appl. No.: 19/454,486

(22) Filed: Jan. 21, 2026

(30) Foreign Application Priority Data
Jan. 23, 2025 (EP) 25153545.6

(51) Int. Cl. A61B 17/32; A61B 17/3203

(52) U.S. Cl. CPC A61B 17/320016; A61B 17/3203; A61B 2017/320064; A61B 2217/005; A61B 2217/007

(57) ABSTRACT

A device for removing tissue particles from the tissue surface or an area close thereto is disclosed. The device comprises a head piece into which a nozzle tube extends with multiple nozzle openings that are oriented toward a head piece opening. Adjacent to the head piece opening the head piece comprises a contact surface for being placed on the tissue surface. Via a supply channel, pressurized liquid can be fed into the nozzle tube and ejected from the nozzle openings to form compact fluid jets to remove tissue particles when impacting on the tissue surface, which are then present in the head piece. The head piece interior space is fluidically connected with a suction channel to suck tissue particles out of the interior space and collect them in a particle collection device. Such a device can be used to gently remove a tissue sample in form of the tissue particles.

### CROSS REFERENCE TO RELATED APPLICATION

This application claims the benefit of European Patent Application No. 25153545.6, filed Jan. 23, 2025, which is incorporated herein by reference in its entirety.

### TECHNICAL FIELD

The invention refers to a device, an arrangement and a method for removing tissue particles from a tissue surface, whereby, for example, cells can be removed or ablated and extracted, particularly as a tissue sample for further pathological analysis. Additionally or alternatively, the invention can also be used for tissue cleaning or tissue treatment on the tissue surface. The invention is particularly suitable for removal of tissue particles on mucous membranes, such as in the stomach, intestine, esophagus, bile duct or other internal tissue surfaces of a human and/or animal body.

### BACKGROUND

The removal of tissue particles or cells close to the surface can be used in order to examine the extracted cells, for example in order to be able to diagnose cell changes at an early stage. Thereby it can be advantageous or necessary to remove or extract tissue particles or cells on larger areas of the tissue surface, so that locally limited tissue or cell changes can be determined with a certain degree of probability. The tissue particles have to be removed from the tissue surface during extraction in order to avoid contamination of other surface areas of the tissue surface, onto which the device acts subsequently. In addition, it must be possible to assign the extracted tissue particles (particularly cells) to a tissue surface or tissue surface area, so that the examination of the cells can be assigned to the tissue surface or tissue surface area from which they have been extracted.

It is known to convey tissue samples into a suction channel by means of a liquid jet in order to subject them to examination. Respective instruments are known from EP 4 072 448 A1, EP 1 182 974 B1, EP 2 019 628 A1, EP 1 433 423 A1, U.S. Pat. No. 6,572,578 B1 and EP 3 500 192 A1. These instruments respectively comprise a fluid channel creating a fluid jet at an outlet opening directly directed onto a suction opening of a suction channel.

Instruments are also known in which a fluid jet is directly directed onto tissue, for example, from EP 2 303 156 A2, US 2021/0308484 A1 and U.S. Pat. No. 6,030,399 A. The latter publication serves for taking blood samples from the skin.

The instrument known from U.S. Pat. No. 6,030,399 A comprises a head that encloses an interior space. The interior space is open toward the surface of the skin from which blood is to be taken. The edge of the opening has to be placed on the skin, whereby the interior space is sealed toward the outside. In order to guarantee that sealing is as complete as possible, the edge of the opening is provided with a corresponding seal. A supply channel serves for supplying liquid into the interior space, whereby the liquid hits the skin in form of a sharp jet and punctures it. Blood or a mixture of blood and supplied fluid, if released in this manner, is drained away via suction channels connected to the interior space. From the technical article Lightdale C J, Tiscornia-Wasserman P, Sethi A et al: Endoscopy-Guided High-Pressure Spray Power-Wash “to obtain Cytopathology For Detection Of Gastric Intestinal Metaplasia: A Proof Of Concept Study”; Gastrointest Endosc 2022; 95: AB457-AB458 it is known that cells can be washed off a mucous membrane of the gastrointestinal tract using a jet.

U.S. Pat. No. 5,037,431 A discloses a surgical device by means of which a fluid jet can be created and can be directed onto a tissue surface in order to fragment diseased tissue. On its distal end the instrument comprises a dome or bell-shaped shield that can be placed on the tissue to be treated. The interior of this shield is additionally connected with a suction line for suction of tissue fragments.

US 2016/199566 A1 describes a hand instrument for cleaning wounds using a water jet. On a hollow cylindrical distal end an axial fluid jet can be produced by means of a fluid nozzle. The face of this hollow cylindrical distal end can be placed on the tissue, whereby the face can be orientated in a plane orthogonal or inclined relative to the cylinder axis. In another embodiment the fluid nozzle is offset from the cylinder axis, whereby the fluid jet hits the tissue surface in a plane of the face of the distal end at the intersection with the cylinder axis.

In the surgical instrument for tissue treatment according to US 2004/0243157 A1 a spherical shield is provided on the distal end limiting an interior space. A suction channel opens out into this interior space. In an axial direction opposite the suction channel a fluid nozzle is arranged, which can direct a fluid jet through the interior space into the suction channel. The shielding is open at one side so that tissue particles project into the interior space of the shield and can be arranged between the fluid nozzle and the suction channel. Thereby the fluid jet hits the tissue portions and can remove parts thereof that are then conveyed directly into the suction channel.

Further instruments for treating tissue with a fluid jet are known from WO 03/096871 A2 and U.S. Pat. No. 6,030,399 A.

### SUMMARY

When extracting tissue samples or when removing or detaching tissue samples from tissue close to the surface, for example in order to examine metaplasia, bleeding tissue injuries should be avoided as far as possible. The invention relates to providing a device or a method with which tissue particles are removed or extracted without damaging the tissue surface and particularly (as far as possible) without causing bleeding tissue injuries.

It can be considered as one object of the present invention to provide a device that allows a gentle and in particular bloodless superficial collection of cells.

This object is solved by means of a device, an arrangement and a method as disclosed herein.

The device according to the invention is configured to remove tissue particles—particularly tissue cells—from a tissue and preferably from a tissue surface. Thereby cell samples or tissue particle samples can be obtained, which can then be collected for examination, for example. Thereby, the device is configured to avoid affecting the tissue or tissue surface more than necessary and to particularly avoid bleeding injuries. The tissue particles or cells can be taken from a human or animal body. For example, the tissue can be mucous membrane—for example in the stomach, intestine, esophagus or bile duct—or another internal tissue surface.

The device uses multiple liquid jets that are directed on the tissue or the tissue surface and impact there. Due to the impact, tissue particles or cells are detached that can be subsequently sucked up by the device and can be collected in a particle collection device. Water or physiological saline solution can be used as liquid for producing the liquid jets, for example.

For this purpose, the device has a head piece having an interior space that is accessible from the outside via a head piece opening. Apart from the head piece opening, the head piece separates the interior space from the direct environment. Optionally one or more ventilation openings can connect the direct environment of the head piece fluidically with the interior space in addition to the head piece opening, in order to allow a gas flow into the interior space.

The head piece has a contact surface configured to be placed on the tissue or the tissue surface during the use of the device. The contact surface is arranged adjacent to the head piece opening and can directly adjoin the head piece opening. In an embodiment the contact surface can be at least present on two opposite sides of the head piece opening of the head piece. In particular, the contact surface can limit the head piece opening on two or more sides and can, for example, surround the head piece opening entirely. In an embodiment the contact surface can extend in a plane that is orientated parallel to a longitudinal direction and parallel to a transverse direction. Alternatively, the contact surface can be orientated along a virtual curved surface, particularly a virtual cylindrical surface. This curved virtual surface is preferably curved in transverse direction and straight in longitudinal direction.

The device comprises a nozzle tube and/or a nozzle hose on the head piece. The nozzle tube and/or nozzle hose extends along the longitudinal axis with distance to the head piece opening into the interior space. Multiple nozzle openings are provided on the nozzle tube and/or nozzle hose. It is preferred that the nozzle openings are arranged along a straight line that is orientated parallel to the longitudinal direction. The nozzle openings are provided on the side of the nozzle tube and/or nozzle hose facing the head piece opening. The nozzle openings are directed toward the head piece opening.

The nozzle openings are preferably oblong or slit-shaped and have a larger dimension (length) in longitudinal direction compared with their dimension in the transverse direction (width).

A supply channel is fluidically connected with the interior of the nozzle tube or nozzle hose and thus with the nozzle openings. Via the supply channel the nozzle tube or nozzle hose is fluidically connected or connectable with a liquid source. Thus, pressurized liquid can be supplied via the supply channel into the nozzle tube and/or the nozzle hose and can be ejected through the nozzle openings of the nozzle tube or nozzle hose.

Each nozzle opening is configured to form a preferably compact, continuous liquid jet if the nozzle tube and/or the nozzle hose is supplied with pressurized liquid. This liquid jet exits the respective nozzle opening, passes through a section of the interior space of the head piece and can then, via the head piece opening, impact on tissue or tissue surface arranged adjacent thereto, particularly if the contact surface of the head piece abuts against the tissue. The liquid pressure inside the nozzle tube or nozzle hose and/or at the liquid source can be, for example, minimum 1.0 to 80 bar and preferably maximum 10 bar. Thus, the liquid exiting the nozzle openings is not atomized.

Preferably, the nozzle openings are orientated, so that the liquid jets are orientated orthogonal to the longitudinal axis and further preferably orthogonal to the transverse direction. The nozzle openings are preferably orientated, so that the liquid jets are orientated parallel to each other and are particularly located in a common plane containing the longitudinal axis. The liquid jets preferably pass through the plane of the head piece opening with orthogonal orientation. Preferably, the orientation of the liquid jets relative to the longitudinal direction and/or the transverse direction can be in an angular range of including 85° to including 95° or of including 87° to including 93° or of including 89° to including 91°.

A suction channel also opens into the interior space. Thus, the suction channel is fluidically connected with the interior space. If the suction channel creates a suction flow, for example in that the suction channel is connected to a suction unit or vacuum unit, the tissue particles or tissue cells removed from the tissue by the liquid jets can be sucked out of the interior space and fed to a particle collection device. Thereby the tissue particles can be conveyed using a fluid flow inside the suction channel. Also, a gas and/or a liquid can be contained in this fluid flow or suction flow, for example parts of the liquid ejected by the device, tissue liquid, air or gas from the environment of the head piece.

By means of the multiple liquid jets, tissue particles or tissue cells can be concurrently removed at multiple positions of the tissue and collected in this manner. When the head piece is moved over the tissue surface, tissue samples of larger tissue areas can be collected quickly and efficiently. For example, this can be of importance if larger tissue areas in an animal or human body shall be examined for tissue or cell changes and for this purpose tissue samples have to be taken at several different locations along the entire tissue area.

Preferably, the suction channel opens out in the interior space adjacent to the nozzle tube and/or nozzle hose. The suction channel can comprise one or more channel openings. For example, multiple channel openings of the suction channel can be arranged in circumferential direction around the longitudinal axis in a distributed manner around the nozzle tube and/or nozzle hose. The at least one channel opening is orientated in a plane orthogonal or obliquely to the longitudinal direction, for example. In the area of the channel openings the suction flow is orientated at least substantially parallel to the longitudinal axis.

In addition, the device comprises an outer shell, which can also be denoted as shank. The outer shell is preferably flexibly bendable. The outer shell is preferably connected directly to the head piece. Alternatively, also individual connection parts can be present between the head piece and the outer shell. In the preferred embodiment the outer shell is a hose that can be bent transverse to its extension direction. The bendability of the hose is so that it can be elastically bent during endoscopic use of the device by the forces that are usually applied on the hose by the endoscope. The outer shell limits a lumen, preferably one single continuous lumen, that extends along the outer shell from the proximal end up to the distal end of the outer shell. The outer shell is hollow cylindrical so to speak.

In a preferred embodiment the head piece does not project—with view orthogonal to the longitudinal axis—at any location beyond a virtual cylindrical surface, wherein the diameter of the virtual cylindrical surface corresponds to the maximum outer diameter of the outer shell. The virtual cylindrical surface can be an extension of the circumferential surface of the outer shell from its distal end or a position with maximum outer diameter in distal direction. Due to this dimensioning, the head piece and the outer shell can be particularly advantageously inserted through the working channel of an endoscope into a body lumen of a human or animal patient.

Alternatively, the head piece can partly or entirely consist of an elastically deformable material, so that it does not project through the virtual cylindrical surface at least in an elastically deformed condition and comprises one or more components which project through the virtual cylindrical surface in the elastically non-deformed initial condition. For example, the head piece can have at least one foot section arranged laterally adjacent to the head piece opening and extending away from the head piece opening in transverse direction or obliquely to the transverse direction. On the at least one foot section the contact surface can be provided, which can be enlarged in transverse direction in this manner. The at least one foot section or the entire head piece can be elastically deformable. In the initial condition the at least one foot section can project through the virtual cylindrical surface and can be elastically deformed out of this initial condition (for example manually) inwards toward the head piece opening and/or above the head piece opening and/or into the head piece opening.

The supply channel and/or the suction channel are preferably provided in the lumen of the outer shell. In an embodiment a supply hose forming the supply channel and fluidically connected with the nozzle tube and/or the nozzle hose is guided through the lumen. It is particularly preferred that the suction channel is directly limited at least by a section of the outer shell and thus the suction channel is formed by at least a section of the lumen, for example by means of a portion of the lumen that is ring-shaped in a cross-section through the outer shell.

It is advantageous if the head piece is rotatably supported around a rotation axis. The rotation axis can correspond to the longitudinal axis or can be arranged offset parallel to the longitudinal axis. In an embodiment, the head piece can be rotatably supported in a passive or uncontrolled manner, so to speak, by the forces acting on the head piece from outside. In this embodiment no operating device is provided on the device that is able to influence the rotation position of the head piece around the rotation axis. Alternatively, a rotation connection device can be present in order to be able to specifically adjust the rotation position of the head piece using a proximately provided operating element.

In an embodiment in which the rotation position of the head piece can be controlled, a rotation connection member is provided from the head piece to an operating element, wherein the rotation connection member preferably extends through the lumen of the outer shell. For example, the operating element can be arranged on a manual operating part of the device from which the outer shell extends up to the head piece. The rotation connection member is configured to transmit a torque from the operating element onto the head piece, in order to modify or adjust its rotation position around the rotation axis. The rotation connection member can be connected with its proximal end in a torque-proof manner with the operating element and with its distal end in a torque-proof manner with the head piece, for example.

The rotation connection member, the supply line and the outer shell are particularly flexibly, elastically bendable transverse to their extension direction when subject to external forces that occur during intended use (for example in connection with an endoscope).

The outer shell and the supply hose are preferably made of a plastic, for example silicone, polyurethane, a thermoplastic elastomer, particularly a thermoplastic polyamide elastomer, which are particularly configured, so that the above-mentioned elastic bendability is provided transverse to their extension direction.

The rotation connection member can comprise, for example, one or more layers of respectively one or more helically wound and/or braided and/or twisted wires (for example, as known from steel cables), whereby an elastic bendability transverse to its extension direction can be realized, for example.

The rotation connection member can optionally also be configured as laser-structured tube. The structure is particularly such that the laser-structured tube is bendable or flexible under the forces occurring during intended use. The laser-structured tube may be torsionally rigid under the forces occurring during intended use, so that no or no significant torsion occurs. Optionally, the tube can be sheathed to accommodate the fluidic supply line (water jet).

The rotation connection member can be configured with or without passage channel extending along the rotation connection member. If the rotation connection member comprises a passage channel, the supply channel can extend inside the passage channel of the rotation connection member, for example the supply hose can be guided through the passage channel.

Alternatively, it can also be advantageous to arrange the rotation connection member in the supply channel and to guide it through the supply hose, for example. In this embodiment the rotation connection member is preferably configured without passage channel and a substantially solid body so to speak. Thereby smaller interstices can be provided in the rotation connection member if the latter is formed by twisting, braiding or otherwise connecting multiple wires.

For rotatability of the head piece a rotary bearing can be created between the outer shell and the head piece, preferably a bearing extension of the head piece. For example, the bearing extension of the head piece can protrude into the distal end of the outer shell or the outer hose. In this manner a rotary bearing in form of a friction bearing can be formed between the head piece and the outer shell. In doing so, a simple and inexpensive rotational support can be realized. Such a rotational support allows to configure the device in a compact manner in the area of the head piece, so that the dimensions radial to the longitudinal axis can be kept small. In this configuration the device is very well suited for use in combination with an endoscope.

At least one fluid passage is provided on the bearing extension of the head piece, particularly for fluidical connection of the interior space with the suction channel. By means of the fluid passage, the bearing extension is configured for guiding a fluid flow therethrough. The at least one fluid passage can be fluidically branched or unbranched and can, for example, be fluidically divided in sections by means of support elements of the bearing extension arranged between them, for example.

It is preferred that the nozzle tube and/or the nozzle hose are immovably arranged relative to the head piece. Particularly, the nozzle tube and/or the nozzle hose cannot be rotated or is not rotatable relative to the head piece around the longitudinal axis. In embodiments in which the head piece can be rotated around the rotation axis, the nozzle tube and/or the nozzle hose rotates together with the head piece, so that the orientation of the nozzle openings toward the head piece opening is maintained.

The device can be advantageously used together with an endoscope. For this purpose, the endoscope can comprise a working channel, for example, through which the head piece and the outer shell of the device can be passed. In the area of the proximal end the supply channel can be connected to a liquid source and the suction channel can be connected to a collection device, wherein the collection device can comprise a suction unit or vacuum unit in order to create the suction flow.

Any embodiment of the device or the arrangement of an endoscope with such a device described above can be used as follows:

The head piece is placed with its contact surface on a tissue surface. In doing so, the interior space in the head piece can be at least substantially separated from the environment of the head piece together with the tissue surface in order to collect tissue particles there and discharge them subsequently. For removal of tissue particles from the tissue surface or an area close to the surface of the tissue, pressurized liquid is conveyed to the nozzle tube or nozzle hose. In doing so, one liquid jet is produced at each nozzle opening of the nozzle tube or nozzle hose, which first passes a section of the interior space and then impacts on the tissue via the head piece opening. In doing so, tissue particles or cells are removed there, which are then present in the interior space of the head piece. The tissue particles can then be conveyed out of the interior space and further via the suction channel to a particle collection device by producing a suction flow.

The creation of the liquid jets and the creation of the suction flow can be carried out concurrently or at least partly, temporally overlapping or temporally sequentially.

During the creation of the liquid jets and/or during the creation of the suction flow the head piece can be moved along the tissue surface. It is also possible to carry out the movement of the head piece only if no liquid jets are created and/or if no suction flow is created.

### BRIEF DESCRIPTION OF THE DRAWINGS

Advantageous embodiments of the invention are derived from the dependent claims, the description and the drawing. In the following, embodiments of the invention are explained in detail based on the attached drawing. The drawing shows:

FIG. 1 a schematic illustration of an arrangement comprising an endoscope with a device according to the invention during an exemplary use on a patient,

FIG. 2 a schematic basic illustration of an embodiment of a device according to the invention,

FIG. 3 an embodiment of a head piece of a device according to the invention in a view perpendicular to a head piece opening,

FIG. 4 an embodiment of the head piece of FIG. 3 in a longitudinal section along a longitudinal axis of the head piece,

FIG. 5 a cross-section along the section line A-A in FIG. 4 through a distal end section of an outer shell connected with the head piece,

FIG. 6 an illustration of an embodiment of a manual operating part of the device according to FIG. 2 in a longitudinal section,

FIG. 7 an embodiment of a head piece and a distal end region of an outer shell connected to the head piece in a longitudinal section,

FIG. 8 another embodiment of the manual operating part of the device according to FIG. 2 in a longitudinal section,

FIG. 9 another embodiment of a head piece as well as a distal end section of an outer shell of the device and

FIG. 10 a cross-section through the head piece of FIG. 9 along the section line B-B in FIG. 9.

### DETAILED DESCRIPTION

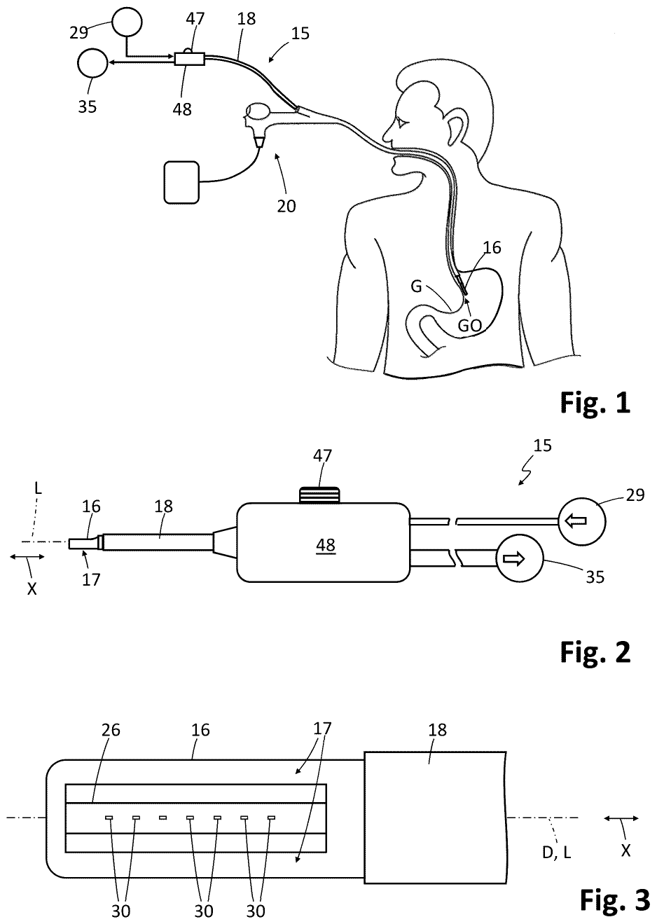

The present invention refers to a method and a device 15 that is configured to remove and collect tissue particles P of a tissue G, particularly from a tissue surface GO or an area of the tissue G close to the surface. The device 15 is schematically apparent from the basic illustrations in FIGS. 1 and 2.

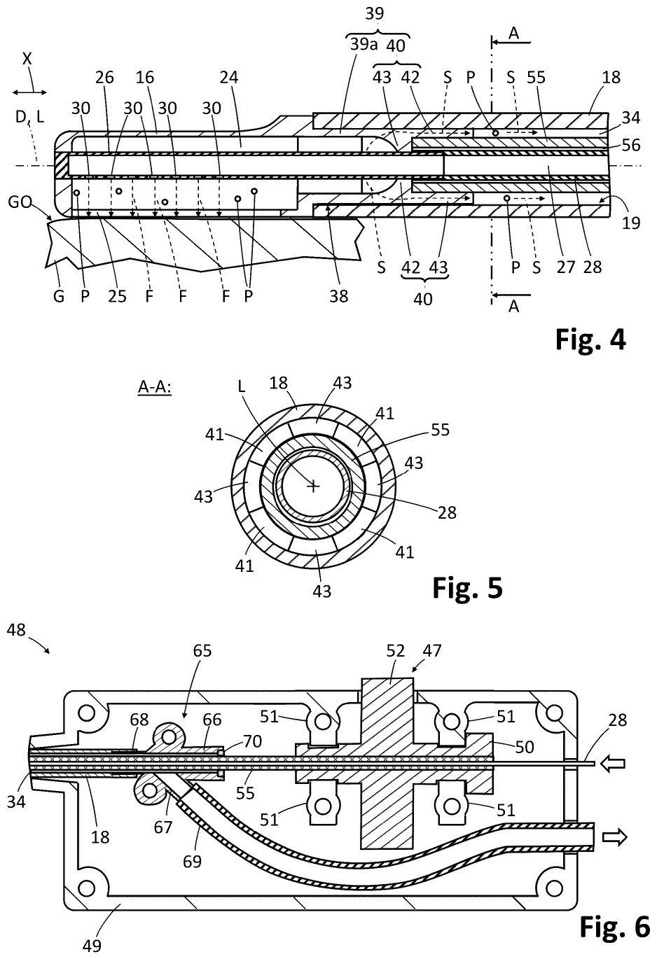

The device 15 has a head piece 16 having a contact surface 17 that is configured to be placed on the tissue surface GO. The head piece 16 is arranged on a distal end of an outer shell 18. The outer shell 18 is formed by a hose, according to the example. A continuous lumen 19 extends through the outer shell 18 from the proximal end up to the distal end of the outer shell 18 (FIGS. 4, 7 and 9). The outer shell 18 is elastically bendable transverse to its extension direction and flexible in such a way that it can be bent by forces typically occurring during the intended use of the device 15, particularly by forces applied on the outer shell 18 by an endoscope, if the outer shell 18 extends through a working channel of the endoscope 20 (FIG. 1).

An arrangement consisting of the endoscope 20 and the instrument 15 is depicted in the exemplary basic illustration in FIG. 1. By means of device 15, preferably in combination with endoscope 20, tissue particles P of a tissue G or a tissue surface GO can be removed and collected. The tissue G can be a mucous membrane, such as the gastric mucosa, for example.

The head piece 16 limits an interior space 24 that is open to the outside on one side by means of a head piece opening 25 and is thus accessible from the outside. Preferably, apart from the head piece opening 25, head piece 16 is otherwise closed relative to the environment of the head piece (FIGS. 4, 7, 9 and 10). Alternatively to this, also at least one ventilation opening (for example through hole) can be present that fluidically connects the interior space 24 with the environment of head piece 16, in order to allow a fluid flow from the environment into the interior space 24.

A nozzle tube 26 extending along a longitudinal axis L protrudes into the interior space 24. The nozzle tube 26 can completely pass through the interior space 24 in a longitudinal direction X (parallel to longitudinal axis L) and can, for example, be connected to the head piece 16 at both ends. The nozzle tube 26 is particularly torque-proof and preferably immovably connected to the head piece 16, for example in a form-fit manner and/or force-fit manner and/or substance-bond manner and/or by means of an adhesive bond. On the face on the distal end the nozzle tube 26 is closed. On the proximal end, nozzle tube 26 is fluidically connected with a supply channel 27. A supply hose 28 serves as supply channel 27 in the embodiment. Analog to the outer shell 18, supply hose 28 is flexibly bendable transverse to its extension direction.

In all embodiments as an alternative to nozzle tube 26 also a nozzle hose or a combination of a nozzle tube 26 and a nozzle hose can be used. In the embodiments indicated here a nozzle tube 26 is used.

The outer shell 18 and the supply hose 28 are preferably made of a plastic, for example silicone, polyurethane, a thermoplastic elastomer, particularly a thermoplastic polyamide elastomer.

The supply channel 27 can be fluidically connected to a liquid source 29 by means of which pressurized liquid can be conveyed through the supply channel 27 or supply hose 28 into the nozzle tube 26.

The nozzle tube 26 has multiple nozzle openings 30, whereby, for example, at least 3 or at least 5 and preferably at most 8 or at most 12 nozzle openings can be provided. The number of nozzle openings allows a good compromise between a fluid ejection or scanning that is as extensive as possible on one hand and a limitation of the required volume flow rate on the other hand.

The nozzle openings 30 are arranged with distance to each other in a longitudinal direction X parallel to the longitudinal axis L. The distance between two directly adjacent nozzle openings is, for example, 0.5 mm to 1.0 mm. The dimension of the nozzle openings 30 in longitudinal direction X (length) is preferably greater than in the transverse direction Q (width). The nozzle openings 30 therefore have an oblong shape, for example a slit shape.

The longitudinal direction X and the transverse direction Q are orientated orthogonal to each other.

According to the example the nozzle openings 30 are arranged in longitudinal direction X along a straight line and thus in one single row. The nozzle openings 30 are arranged on the side of the nozzle tube 26 facing the head piece opening 25. If the interior of nozzle tube 26 is supplied with pressurized liquid via supply channel 27, the liquid exits the nozzle openings 30, so that a compact fluid jet F forms at each nozzle opening 30 and is ejected in direction toward the head piece opening 25 (depicted in FIG. 4 by way of example).

The fluid jets F are preferably orientated parallel to each other. In the embodiment the fluid jets F are arranged in a common plane in which also the longitudinal axis L extends. Through the head piece opening 25 the fluid jets F can be directed onto the tissue surface GO if the head piece 16 is placed onto the tissue surface GO of the tissue G using the contact surface 17 as schematically illustrated in FIG. 4.

In the embodiment the contact surface 17 is arranged directly adjacent to the head piece opening 25. The contact surface 17 can limit the head piece opening 25 at multiple locations and/or on multiple sides. Preferably, the contact surface 17 continuously extends circumferentially around the head piece opening 25 (FIG. 3).

In addition, interior space 24 is fluidically connected with a suction channel 34. In the embodiment suction channel 34 can be limited by outer shell 18 and can be provided in the lumen 19 of outer shell 18, for example in form of a ring channel surrounding the supply hose 28 (FIGS. 4, 7 and 9). At its proximal end suction channel 34 is fluidically connectable to a particle collection device 35 (FIGS. 1 and 2). The particle collection device 35 can comprise a suction unit or vacuum unit in order to produce a suction flow S from the interior space 24 through the suction channel 34 into the particle collection device 35 (FIG. 4). By means of this suction flow S, tissue particles P or tissue cells removed from the tissue G or the tissue surface GO can be conveyed out of the interior space 24 via suction channel 34 into the particle collection device 35 and can be collected there. In the particle collection device one or more particle collection containers can be provided, so that the tissue particles P collected in one particle collection container can be assigned to a specific location of the tissue G or tissue surface GO from which the tissue particles P have been removed and collected.

The term “proximal” defines a direction or position in relation to the device 15 or a part thereof toward the fluid source 29 and/or the particle collection device 35. The term “distal” defines a direction or position in relation to the device 15 or a part thereof away from the fluid source 29 and/or the particle collection device 35. The terms “proximal” and “distal” are not only used in relation to the entire device 15, but also during the description of individual components thereof in relation to the respectively described component.

The device 15 described so far can be used as follows for removal of tissue particles P:

With the contact surface 17 the head piece 16 is placed on a tissue surface GO of a tissue from which tissue particles P shall be removed. By supplying pressurized liquid F from the liquid source 29 via supply channel 27 into nozzle tube 26, a liquid jet F is created at each nozzle opening 30 that exits the nozzle opening 30, passes through a section of the interior space 24 and impacts on the tissue surface GO through head piece opening 25. On the tissue surface GO tissue particles P or tissue cells are detached or removed in a gentle and particularly bloodless manner and are then at first present in the interior space limited by head piece 16. When a suction flow S is produced by means of a suitable suction unit or vacuum unit, the loose tissue particles P are conveyed from the interior space 24 in the suction channel 34 and from the suction channel 34 into the particle collection device 35. Together with the liquid the loose tissue particles P can form a suspension, so to speak. There the tissue particles P can be collected in one or optionally multiple particle collection containers, so that they are available, for example for a further tissue analysis.

Additionally or alternatively, tissue cleaning or another tissue influence of the tissue surface GO or of layers close to the surface of tissue G can be carried out by means of device 15.

In the preferred embodiments head piece 16 is rotatably supported on the outer shell 18 around a rotation axis D. The rotation axis D preferably corresponds to the longitudinal axis L. For this purpose, a rotary bearing 38 can be formed, that is particularly configured as plain bearing, between the head piece and the outer shell 18.

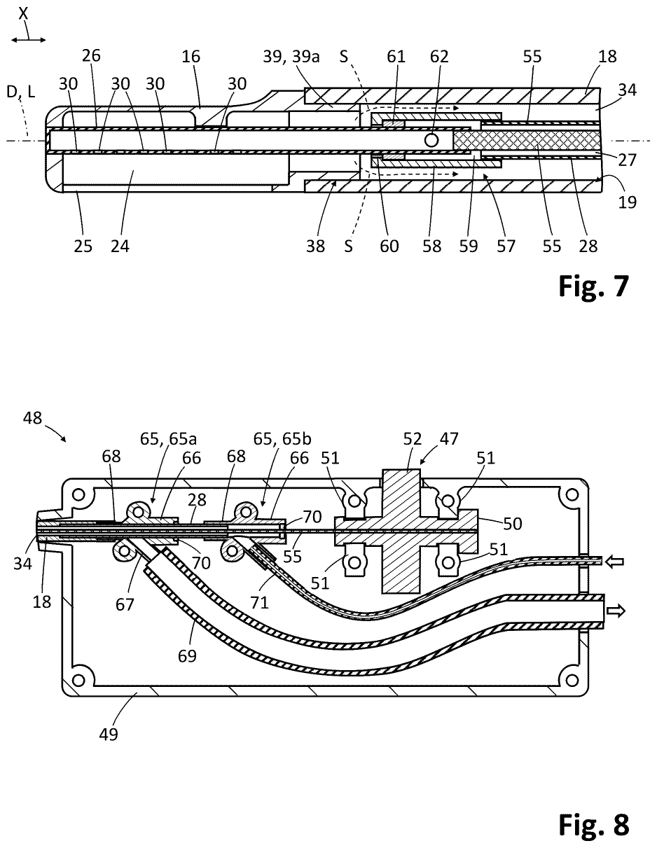

In the embodiments according to FIGS. 4 and 7, head piece 16 comprises a bearing extension 39 whose circumferential surface surrounds the rotation axis D and according to the example the longitudinal axis L coaxially. The bearing extension 39 is inserted in the distal end of outer sleeve 18 and slidingly abuts with its circumferential surface rotatably against the inner surface of outer shell 18. For this, a suitable material pairing and dimensioning is selected. Due to the abutment of outer sleeve 18 on bearing extension 39, the ring-shaped suction channel 34 can be at least substantially closed in fluid-tight manner toward the environment, which is, however, not absolutely necessary. If at this position upon occurrence of a suction flow S, gas is sucked in from the environment surrounding the head piece 16, this is uncritical and can be advantageous in order to prevent sticking of the head piece 16 to the tissue surface GO and to facilitate the displacement of the head piece 16 along the tissue surface GO during the creation of the suction flow S. As already mentioned, for avoiding such sticking, also at least one ventilation opening can be present on the head piece 16, which fluidically connects the interior space 24 with the environment.

In the embodiments illustrated in FIGS. 4 and 7, the bearing extension 39 has a hollow cylindrical part 39a and can be exclusively formed by the hollow cylindrical part 39a, as in the embodiment according to FIG. 7. In contrast thereto the bearing extension 39 can have multiple support elements 40 in the embodiment according to FIG. 4 that are arranged in a circumferentially distributed manner around longitudinal axis L. In doing so, between two support elements 40 directly adjacent in circumferential direction around longitudinal axis L, one fluid passage 41 is formed respectively, in order to provide a fluid connection between the suction channel 34 and the interior space 24. The support elements 40 adjoin the hollow cylindrical part 39a of bearing extension 39 in longitudinal direction X. In the transition area toward the hollow cylindrical part 39a of bearing extension 39, each support element 40 has a radial projection 42 projecting radially inwardly toward the longitudinal axis L according to the example (FIG. 4). A longitudinal web 43 adjoins the radial projection in longitudinal direction X. The outer surface of each longitudinal web 43 facing away from longitudinal axis L extends in longitudinal direction X in extension to the circumferential surface of the hollow cylindrical part 39a of bearing extension 39.

In the embodiment according to FIGS. 4 and 7, head piece 16 together with nozzle tube 26 arranged thereon can be rotated by means of an operating element 47 on a manual operating part 48 of device 15. By means of operating element 47 a rotational movement or torque can be applied on head piece 16 in order to change and adjust its rotational position around the rotation axis D or longitudinal axis L. The manual operating part 48 having the operating element 47 is apparent from FIGS. 1, 2, 6 and 8. It has a housing 49 on which the operating element 47 is rotatably supported. For this purpose, operating element 47 comprises a swivel pin 50 that is rotatably supported at two distanced positions on swivel bearing parts 51 of housing 49. According to the example the swivel bearing parts 51 are arranged in the interior of housing 49. On swivel pin 50 a swivel disc or swivel ring 52 of operating element 47 is arranged in torque-proof manner and projects at least at one position out of housing 49 via a housing opening. At this position the operating element 47 or swivel ring 52 is accessible, so that the operating element 47 can be rotated around the axis of swivel pin 50. In the embodiment swivel ring 52 is formed by means of a closed disc, however can also be connected to swivel pin 50 via spoke-like connections.

For transmission of the rotation movement of operating element 47 to head piece 16 a rotation connection is provided extending through lumen 19 of outer shell 18, wherein the rotation connection is realized by rotation connection member 55 according to the example. The rotation connection member 55 is elastically bendable transverse to its extension direction. For example, it can comprise one or multiple helically wound wires. Multiple of such wires can be interwoven, twisted or connected in another appropriate manner. The rotation connection member 55 can comprise multiple layers of one or more helically wound wires respectively.

The rotation connection member 55 is connected with operating element 47 in torque-proof manner at its proximal end and according to the example with swivel pin 50. When rotating operating element 47, rotation connection member 55 is rotated around its center axis and transmits this rotation movement to head piece 16. For this purpose, the distal end of rotation connection member 55 is connected in torque-proof manner with head piece 16.

In the embodiment illustrated in FIG. 4 the distal end of rotation connection member 55 is directly connected with head piece 16 in torque-proof manner, according to the example with bearing extension 39. For this purpose, the support elements 40 of bearing extension 39 project over the distal section of rotation connection member 55. Thereby the radial projections 42 as well as the longitudinal webs 43 of support elements 40 can abut against rotation connection member 55 as schematically shown in FIG. 4.

In modification to this the rotation connection member 55 can also be directly attached to nozzle tube 26 in torque-proof manner (FIG. 7), for example, the distal end of the rotation connection member 55 can project in the proximal end of nozzle tube 26 and can be attached there in force-fit manner and/or in substance-bond manner and/or in form-fit manner and/or using an adhesive bond.

In the embodiment illustrated in FIG. 7 the rotation connection member 55 has a tube-shaped or a hose-shaped form having a passage channel 56 passing through the rotation connection member 55 from the proximal end up to the distal end. The supply channel 27 can extend through this passage channel 56. For example, the supply hose 28 can be guided through the passage channel 56 as apparent from FIG. 4.

In the embodiment illustrated in FIG. 4 the supply hose 28 comprising the supply channel 27, the rotation connection member 55 and the outer shell 18 are arranged coaxially relative to each other. Between the rotation connection member 55 and the outer shell 18, the suction channel 34 is provided that is divided into fluid passages 41 by the longitudinal webs 43 of support elements 40 in the distal region of the outer shell. Each fluid passage 41 opens in the interior space 24 adjacent to nozzle tube 26. Thus, multiple channel openings can be arranged around nozzle tube 26 distributed in circumferential direction around longitudinal axis L.

In contrast to the embodiment according to FIG. 4 the rotation connection member 55 is configured without passage channel in the embodiment according to FIG. 7. In this configuration the rotation connection member 55 is centrally connected with nozzle tube 26 in extension to the longitudinal axis L in the distal end section of outer shell 18 and thus centrally arranged in the outer shell 18 or in the lumen 19. The support hose 28 is coaxially arranged around rotation connection member 55. In this embodiment the supply channel 27 is formed in ring-shaped manner around rotation connection member 55 inside supply hose 28.

For providing a fluid connection to nozzle tube 26, a coupling device 57 is provided according to the example, which comprises a hollow cylindrical coupling sleeve 58. The coupling sleeve 58 is connected to the distal end of supply hose 28 in fluid-tight manner. For example, the supply hose 28 can be inserted with its distal end in coupling sleeve 58 and glued, welded or otherwise attached there in fluid-tight manner. The coupling sleeve 58 surrounds a fluid chamber 59 into which the proximal end of nozzle tube 26, which is connected to the rotation connection member 55, through a face-side opening 60.

In the fluid chamber 59 adjacent to the face-side opening 60 a ring seal 61 is arranged. The ring seal 61 also abuts against nozzle tube 26. The ring seal 61 also abuts against a wall section of coupling sleeve 58 adjacent to the face-side opening 60. In this manner ring seal 61 seals the fluid chamber 59 against the outer side of nozzle tube 26 and against the face-side opening 60. Inside the section extending into the fluid chamber 59 nozzle tube 26 has one or more inlet openings 62. Through the inlet openings a liquid supplied via supply channel 27 into fluid chamber 59 can enter the interior of nozzle tube 26 and can then be ejected through the nozzle openings 30 for forming the liquid jets F.

If operating element 47 is rotated the rotation is transmitted on rotation connection member 55, which transmits the rotation in turn on head piece 16, either directly (FIG. 4) or indirectly by means of nozzle tube 26 (FIG. 7).

In the embodiment according to FIG. 4 the supply hose 28 lies without torque-proof connection inside passage channel 56 of rotation connection member 55. Due to the connection of the supply hose 28 with nozzle tube 26, the distal end of supply hose 28 is also rotated, wherein the proximal end of supply hose 28 is not rotated in the preferred embodiment. Due to the length of supply hose 28 from the proximal end to the distal end, the torsion of supply hose 28 created thereby can be tolerated. The torsion of supply hose 28, in case of a single complete rotation of head piece 16 starting from a non-torsioned rest position of supply hose 28, is small and therefore uncritical.

In the embodiment according to FIG. 7 nozzle tube 26 is rotatably supported by means of coupling device 57 and can rotate relative to the ring seal 61 of coupling device 57. Thus, supply hose 28 remains in a non-torsioned or non-rotated position independent from the rotation position of head piece 16.

In a section between the proximal end of outer shell 18 on one side and the liquid source 29 or particle collection device 35 on the other side, the device 15 can comprise at least one fluid coupling element 65. Each fluid coupling element 65 is configured to transfer two components of the device 15 (for example supply channel 27 or supply hose 28, suction channel 34, rotation connection member 55) coaxially arranged inside lumen 19 of outer shell 18 into a non-coaxial arrangement while maintaining the fluid tightness of a respective channel (for example, supply channel 27, suction channel 34).

As already explained, liquid source 29 and particle collection device 35 (including a suction unit or vacuum unit) are connected to the supply channel 27 or the suction channel 34 respectively via housing 49 of manual operating part 48. By means of the at least one fluid coupling element 65 the fluid connections can be established as shown based on FIGS. 6 and 8. The at least one fluid coupling element 65 can be arranged in the inside of housing 49.

For connecting the supply hose 28 arranged inside passage channel 56 of rotation connection member 55 (FIGS. 4 and 6) the supply hose 28 can be led out of the proximal end of rotation connection member 55 and can be connected by means of a suitable fluid connection with liquid source 29. For example, for this purpose, the swivel pin 50 can comprise a through hole extending along its longitudinal axis through which the supply hose 28 extends. Inside the through hole the rotation connection member 55 can be connected in torque-proof manner to the swivel pin 50 (FIG. 6).

In the embodiment illustrated in FIG. 6 one single fluid coupling element 65 is sufficient. The fluid coupling element 65 has a first inlet channel 66, a second inlet channel 67 and an outlet channel 68. The rotation connection member 55 as well as the supply line 28 arranged therein extend into the first inlet channel 66 and out of the outlet channel 68. The outer shell 18 is connected to the outlet channel 68 in fluid-tight manner. Adjacent to the outlet channel 68 supply hose 28, rotation connection member 55 and outer shell 18 are arranged coaxially with each other.

The ring-shaped suction channel 34, which is present between outer shell 18 and rotation connection member 55, is fluidically connected with second inlet channel 67 in the fluid coupling element 65. The second inlet channel 67 of fluid coupling element 65 is fluidically connected with a suction hose 69, which can be let out of the housing 49 and can be connected to the particle collection device 35. In order to seal the suction channel 34 against the interior of housing 49, a fluid seal 70 is arranged on first inlet channel 66 that abuts on the outside against rotation connection member 55 in fluid-tight manner. The rotation connection member 55 is rotatable relative to fluid seal 70. For example, fluid seal 70 can be an O-ring.

The embodiment of manual operating part 48 illustrated in FIG. 8 comprises the same fluid coupling element 65 for fluid connection of suction hose 69 with suction channel 34 as it has been explained above in connection with FIG. 6.

In a device 15, in which the rotation connection member 55 is arranged inside supply hose 28 (FIG. 7), an additional fluid coupling element 65 is provided in the housing 49 as shown in FIG. 8. For sake of distinction, in FIG. 8 the fluid coupling element 65, which is also present in the embodiment according to FIG. 6, is denoted as first fluid coupling element 65a, whereas the additional fluid coupling element 65 is denoted as second fluid coupling element 65b.

The second fluid coupling element 65b is proximally arranged to the first fluid coupling element 65a. The second fluid coupling element 65b has essentially the same configuration as the first fluid coupling element 65a described above and comprises a first inlet channel 66 having a fluid seal 70, a second inlet channel 67 and an outlet channel 68. Through the first inlet channel 66 the rotation connection member 55 is guided in fluid-tight manner and exits through outlet channel 68. Connected in fluid-tight manner with the outlet channel 68 is supply line 28, in which the rotation connection member 55 is inserted, so that the supply line 28 contains the rotation connection member 55 inside supply channel 27 adjacent to the outlet channel 68. This coaxial arrangement can then be guided into the first inlet channel 66 of first fluid coupling element 65a.

Via second inlet channel 67 of second fluid coupling element 65b the supply channel 27 inside supply hose 28 is fluidically connected to a fluid line 71. Starting from the second inlet channel 67 the fluid line 71 is led out of the housing 49 and can be connected to the liquid source 29 using suitable connection means.

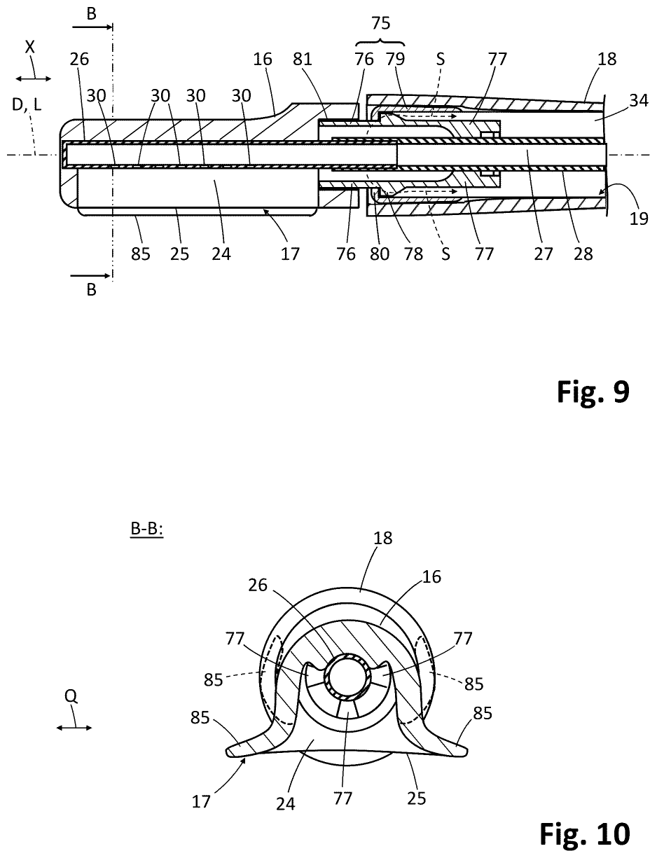

In the embodiments of device 15 explained so far, the head piece 16 is actively controllable or adjustable with regard to its rotational position around the rotation axis D or longitudinal axis L. In modification to this, the head piece 16 can be rotatably supported on the distal end of outer shell 18 without rotational connection to the proximal end, so that the rotation connection member 55 guided through the lumen 19 of outer shell 18 can be omitted. The rotation position of head piece 16 is then defined by forces acting on the head piece 16 from outside, for example during the movement of the head piece 16 along a tissue surface GO or also due to forces created during production of the suction flow S and/or the fluid jets F. Such an embodiment is schematically illustrated in FIGS. 9 and 10.

In all embodiments the head piece 16 can consist of multiple parts and/or can comprise different materials in different sections. For example, the bearing extension 39 can consist of a different material or can contain a different material than the remaining part of the head piece 16.

In the embodiment illustrated in FIGS. 9 and 10 the head piece 16 does not have a bearing extension 39. The head piece 16 is indirectly connected with the distal end of outer shell 18 by means of a connection device 75. The connection device 75 has a connection sleeve 76 that can be configured similarly to the bearing extension 39 on head piece 16 according to FIGS. 4 and 5. The connection sleeve 76 can particularly comprise a hollow cylindrical part and connection elements 77 adjoining thereto in longitudinal direction X, which are arranged with distance to each other in circumferential direction around the longitudinal axis L, analog to the support elements 40, so that fluid passages for the suction flow S are formed therebetween. In the transition area between the hollow cylindrical part and the connection element 77 the connection sleeve 76 has one or more holding projections 78 extending outwardly away from the longitudinal axis L. For example, one single ring-shaped holding projection 78 can be present or multiple holding projections 78 arranged with distance to one another in circumferential direction around the longitudinal axis L can be provided on the connection sleeve 76.

For connection to the distal end the connection device 75 comprises in addition an outer sleeve 79. The outer sleeve 79 limits an interior into which the connection sleeve 76 including the at least one holding projection 78 can be inserted from the side facing away from the head piece 16. On the side facing the head piece 16 the outer sleeve 79 comprises at least one stop 80, which can be formed, for example, by a ring flange or a ring shoulder. Alternatively, also multiple stops can be provided in circumferential direction around the longitudinal axis L extending toward the longitudinal axis L. The stops 80 limit an opening through which the hollow cylindrical part of the connection sleeve 76 can extend. The at least one holding projection 78 abuts against the at least one stop 80. In circumferential direction around the longitudinal axis L the dimensions of the at least one holding projection 78 and the at least one stop 80 are selected so that in each rotational position around the longitudinal axis L pulling the connection sleeve 76 out of the outer sleeve 79 is avoided. For example, this can be achieved in that at least one of the stops 80 or the holding projection 78 is configured in ring-shaped manner.

The outer sleeve 79 is connected with the distal end of outer shell 18, for example in force-fit manner and/or substance-bond manner and/or using an adhesive bond. The connection sleeve 76 is rotatably arranged in the outer sleeve 79. The connection sleeve 76 is connected to the head piece 16 in torque-proof manner and can, for example, be attached in a cavity 81 of head piece 16 in torque-proof manner. By means of the connection device 75 friction forces for rotating the head piece 16 around rotation axis D or longitudinal axis L can be reduced, in that a suitable material pairing is selected for the connection sleeve 76 and the outer sleeve 79. The connection sleeve 76 and the outer sleeve 79 can be made of metal, a metallic alloy, ceramic, or the like, for example. Due to the short length in longitudinal direction X, the connection device 75 does not have to be elastically deformable transverse to the longitudinal direction X.

By way of example a cross-section through the head piece 16 according to section line B-B in FIG. 9 is illustrated in FIG. 10. The configuration of this head piece 16 can be used in all of the embodiments. It is particularly suitable for a head piece 16 that is not rotatable in controlled manner via operating element 47 and only (so to speak passively) rotatably supported on the distal end of outer shell 18.

In the embodiment illustrated in FIG. 10, adjacent to the head piece opening 25, the head piece 16 has one foot section 85 extending orthogonal or obliquely outwardly from the head piece opening 25 on two sides respectively, arranged opposite in transverse direction Q. Via this foot section 85 a laterally or in transverse direction wider and thus enlarged contact surface 17 can be formed.

The foot sections 85 and as an option also at least a section of the head piece 16 joining the foot sections 85 or also the entire head piece 16 can be made of an elastically deformable material in this embodiment, wherein materials can be considered from which also the outer shell 18 and/or the supply hose 28 are made. In doing so, the foot sections can be brought from a non-elastically deformed initial position (solid line in FIG. 10) into an elastically deformed position (dashed line in FIG. 10). In the elastically deformed position, the foot sections can extend over and/or into the head piece opening 25. The foot sections 85 can take this position, for example, in order to move the head piece 16 through the working channel of endoscope 20. Outside of the working channel and without external influence the foot sections 85 assume (again) the non-elastically deformed initial position.

In modification to the illustrated embodiments in all of the configurations of head piece 16, openings can fluidically connect the interior space 24 with the environment in order to avoid a too strong sticking of the head piece on the tissue surface GO during production of the suction flow S.

The invention relates to a device 15 for removal of tissue particles P of a tissue G from the tissue surface GO or a tissue are close to the surface, particularly without causing bleeding. In doing so, tissue particles P or cells can be collected, for example for a subsequent tissue analysis. For this purpose, device 15 comprises a head piece 16 limiting an interior space 24 into which a nozzle tube 26 protrudes. The nozzle tube has multiple nozzle openings 30 arranged adjacent to one another which are orientated toward a head piece opening 25 of the head piece 16. Adjacent to the head piece opening 25 the head piece 16 has a contact surface 17, which is configured for being placed on the tissue surface GO. Pressurized liquid can be fed into the nozzle tube 30 via a supply channel 27 and can be ejected from the nozzle openings 30 forming compact fluid jets in order to remove tissue particles P when hitting the tissue surface GO, which are then present in the interior space 24 of head piece 16. The interior space 24 is fluidically connected with a suction channel 34. A suction flow S can suck tissue particles out of the interior space 24 via the suction channel and collect them in a particle collection device 35, which can be fluidically connected to the suction channel 34. By means of such a device 15, a tissue sample in the form of tissue particles P can be gently removed. The device 15 can be used in combination with an endoscope 20.

### LIST OF REFERENCE SIGNS

15 device
16 head piece
17 contact surface
18 outer shell
19 lumen of outer shell
20 endoscope
24 interior space
25 head piece opening
26 nozzle tube
27 supply channel
28 supply hose
29 liquid source
30 nozzle opening
34 suction channel
35 particle collection device
38 rotary bearing
39 bearing extension
39a hollow cylindrical part of bearing extension
40 support element
41 fluid passage
42 radial projection of support element
43 longitudinal web of support element
47 operating element
48 manual operating part
49 housing
50 swivel pin
51 swivel bearing part
52 swivel ring
55 rotation connection member
56 passage channel
57 coupling device
58 coupling sleeve
59 fluid chamber
60 face-side opening
61 ring seal
62 inlet opening
65 fluid coupling element
65a first fluid coupling element
65b second fluid coupling element
66 first inlet channel
67 second inlet channel
68 outlet channel
69 suction hose
70 fluid seal
71 fluid line
75 connection device
76 connection sleeve
77 connection element
78 holding projection
79 outer sleeve
80 stop
81 cavity of head piece
85 foot section
D rotation axis
F liquid jet
G tissue
GO tissue surface
L longitudinal axis
P tissue particle
Q transverse direction
S suction flow
X longitudinal direction

1. A device (15) for removing of tissue particles (P) from a tissue (G), the device (15) comprising:
a head piece (16) that surrounds an interior space (24) and comprises a head piece opening (25) through which the interior space (24) is accessible from outside the head piece (16) and which is arranged adjacent to a contact surface (17) of the head piece (16), wherein the contact surface (17) is configured for being placed on a tissue surface (GO);
at least one of a nozzle tube (26) and a nozzle hose that extends along a longitudinal axis (L) at a distance to the head piece opening (25) and into the interior space (24) and comprises multiple nozzle openings (30) oriented toward the head piece opening (25);
a supply channel (27) that is fluidically connected with the at least one of a nozzle tube (26) and a nozzle hose and that is configured to guide liquid from a liquid source (29) to the at least one of a nozzle tube (26) and a nozzle hose; and
a suction channel (34) that is fluidically connected with the interior space (24) and that is configured to guide tissue particles (P) from the interior space (24) to a particle collection device (35).

2. The device according to claim 1, wherein the suction channel (34) comprises at least one channel opening into the interior space (24) of the head piece (16), arranged adjacent to the nozzle tube (26), wherein particularly multiple channel openings are arranged in a distributed manner around the nozzle tube (26).

3. The device according to claim 1, wherein the nozzle openings (30) are configured to orient liquid jets (F) exiting the nozzle openings to be parallel to each other and/or orthogonal to the head piece opening (25).

4. The device according to claim 1, wherein the nozzle openings (30) are arranged parallel to the longitudinal axis (L) along a common straight line.

5. The device according to claim 1, further comprising an outer shell (18) connected to the head piece (16), wherein the outer shell (18) defines a lumen (19) extending from a proximal end to a distal end of the outer shell (18).

6. The device according to claim 5, wherein a supply hose (28) is arranged inside the lumen (19), wherein the supply hose (28) comprises the supply channel (27) and wherein the suction channel (34) is arranged inside the lumen (19).

7. The device according to claim 6, wherein the head piece (16) is rotatably supported around a rotation axis (D) that extends parallel to the longitudinal axis (L) or along the longitudinal axis (L).

8. The device according to claim 7, wherein the head piece (16) is passively rotatably supported by forces acting on the head piece (16) from outside the device.

9. The device according to claim 6 wherein inside the lumen (19) a rotation connection member (55) is arranged that is connected with the head piece (16) in a torque-proof manner at a distal end thereof and that is connected with an operating element (47) in a torque-proof manner at a proximal end thereof and that is configured to transmit a torque from the operating element (47) to the head piece (16).

10. The device according to claim 9, wherein the rotation connection member (55) comprises a passage channel (56) through which the supply channel (27) and the supply hose (28) extends.

11. The device according to claim 9, wherein the rotation connection member (55) is arranged in the supply channel (27) and inside the supply hose (28).

12. The device according to claim 7, wherein a rotary bearing (38) is configured to allow rotary movement between the outer shell (18) and a bearing extension (39) of the head piece (16), wherein the bearing extension (39) extends into the lumen (19) of the outer shell (18).

13. The device according to claim 12, wherein the bearing extension (39) comprises at least one fluid passage (41) extending from a proximal end of the bearing extension (39) to the interior space (24) of the head piece (16).

14. The device according to claim 1, wherein the at least one of a nozzle tube (26) and a nozzle hose is immovably arranged relative to the head piece (16).

15. An arrangement comprising:
the device (15) according to claim 1; and
an endoscope (20) having at least one working channel and the device (15).

16. A method for removing tissue particles (P) from a tissue using a device (15), the device (15) comprising:
a head piece (16) that surrounds an interior space (24) and comprises a head piece opening (25) through which the interior space (24) is accessible from outside the head piece (16) and which is arranged adjacent to a contact surface (17) of the head piece (16), wherein the contact surface (17) is configured for being placed on a tissue surface (GO);
at least one of a nozzle tube (26) and a nozzle hose that extends along a longitudinal axis (L) at a distance to the head piece opening (25) and into the interior space (24) and comprises multiple nozzle openings (30) oriented toward the head piece opening (25);
a supply channel (27) that is fluidically connected with the at least one of a nozzle tube (26) and a nozzle hose and that is configured to guide liquid from a liquid source (29) to the at least one of a nozzle tube (26) and a nozzle hose; and
a suction channel (34) that is fluidically connected with the interior space (24) and that is configured to guide tissue particles (P) from the interior space (24) to a particle collection device (35);
wherein the method comprises:
placing the contact surface (17) of the device (15) on the tissue surface (GO);
supplying pressurized liquid to the at least one of a nozzle tube (26) and a nozzle hose, whereby multiple liquid jets (F) are ejected from the nozzle openings (30) and through the head piece opening (25) onto the tissue (G) to remove tissue particles (P); and
sucking at least a part of the removed tissue particles (P) out of the interior space (24) of the head piece (16) via the suction channel (34) to the particle collection device (35).

17. The method of claim 16, wherein placing the contact surface (17) of the device (15) on the tissue surface (G) comprises placing the contact surface (17) on the tissue surface of a mucous membrane.

18. The method of claim 17, wherein the mucous membrane is a mucous membrane of at least one of a stomach, an intestine, an esophagus and a bile duct.

19. The method of claim 16, further comprising examining a plurality of cells of the at least a part of the removed tissue particles (P).

20. The method of claim 19, further comprising moving the contact surface (17) of the device (15) to different positions on the tissue surface (GO) such that the multiple liquid jets (F) remove the tissue particles (P) from the different positions of the tissue surface (GO); and wherein examining the plurality of cells comprises determining a change to one or more cells of the plurality of cells removed from the different positions of the tissue surface (GO).
